(19) United States
(12) Patent Application Publication
(10) Pub. No.: US 2025/0352699 A1
(43) Pub. Date: Nov. 20, 2025

(54) CELL SHEETS AND USES

(71) Applicant: Eureka Biotech Inc., Philadelphia, PA (US)

(72) Inventors: Bin JIANG, Philadelphia, PA (US); Zihao DONG, Melbourne (AU)

(21) Appl. No.: 19/210,390

(22) Filed: May 16, 2025

Related U.S. Application Data
(60) Provisional application No. 63/648,712, filed on May 17, 2024.

(51) Int. Cl. A61L 27/38; A61L 27/54; A61L 27/60; C12N 5/077; C12N 5/0775

(52) U.S. Cl. CPC A61L 27/3895; A61L 27/3834; A61L 27/54; A61L 27/60; C12N 5/0655; C12N 5/0656; C12N 5/0667; A61L 2300/412; A61L 2300/64; A61L 2430/34; C12N 2500/32; C12N 2500/84; C12N 2501/115

(57) ABSTRACT

The present disclosure relates to the generation and use of cell sheets, in particular, stem cell sheets, which can preserve stem cell viability and function, and methods of assembling a medical device, methods of treating a wound, and kits comprising such sheets.

### CROSS-REFERENCE TO RELATED APPLICATIONS

This nonprovisional application claims the benefit and priority, under 35 U.S.C. § 119(e) and any other applicable laws or statues, to U.S. Provisional Patent Application Ser. No. 63/648,712 filed on May 17, 2024, the entire disclosure of which is hereby expressly incorporated herein by reference.

### FIELD

### Technical Field

The present disclosure relates to the generation and use of cell sheets, in particular, stem cell sheets, which can preserve stem cell viability and function. The present disclosure also relates to methods of assembling a medical device comprising such cell sheets, methods of treating a wound by using such cell sheets, and kits comprising the same.

### Background

Mesenchymal Stem Cells (MSCs) are multipotent adult stem cells known for their ability to self-renew and differentiate into various cell types. Initially discovered in bone marrow, subsequent research has identified their presence in multiple tissues, including adipose (fat), dental pulp, umbilical cord blood, and amniotic membrane. MSCs may differentiate into a variety of cell types, such as osteocytes (bone cells), chondrocytes (cartilage cells), myocytes (muscle cells), adipocytes (fat cells), and neurons (nerve cells). Owing to their multifunctional nature and relative ease of acquisition, the potential applications of MSCs in regenerative medicine and tissue engineering have been extensively explored.

MSCs have shown potential in treating various diseases, including applications in regenerative medicine. The ability of MSCs to modulate the immune system has been effective in treating autoimmune diseases like multiple sclerosis and osteoarthritis. The regenerative capacity of MSCs has shown success in repairing heart tissue post-myocardial infarction, aiding in bone and cartilage regeneration, and generally accelerating wound healing. Additionally, MSCs offer potential benefits in treating neurodegenerative disorders through their ability to differentiate into neural cells. These diverse applications underscore the versatility of MSCs in medical treatments, making them a focal point of ongoing research and offering hope for innovative therapies in various medical fields.

MSCs may be isolated from multiple tissues or derived from pluripotent stem cells (e.g., embryonic stem cells and induced pluripotent stem cells). This derivation method relies on specific equipment in a high-standard laboratories (for example, ultra-clean benches, constant temperature humidity incubators, and various high-purity gases and incubators) for preparation, maintenance, and quality control. Ordinary medical institutions do not meet these conditions. Therefore, MSC products for cell therapy must be transported over a distance between experimental and medical institutions. However, under ambient conditions, the MSCs may rupture their organelles, and the nuclear damage may eventually lead to apoptosis. The cell debris can potentially cause severe inflammation post-transplantation.

Two primary methods are employed in cell transportation and application: short-distance transport and long-distance transport. Short-distance transport is limited to a 24-hour window and involves cells maintained in culture flasks with growth media, requiring a 37° C. controlled environment. While practical for small volumes, this method risks cell damage and necessitates a significant recovery period, alongside the challenges of expensive culture media and public safety concerns due to liquid transport. On the other hand, long-distance transport predominantly relies on dry ice delivery, where cells are frozen and transported in cryo-vials. Despite its ability to move large quantities of cells, this method incurs additional costs and logistical complexities, including regular dry ice replenishment and the need for specialized transport approvals. Moreover, the risk of dry ice depletion during transit poses a severe threat of irreversible cell damage.

A method called “spheropreservation” for stem cells allows for the storage of human mesenchymal stem cells (hMSC) in spheroids, preserving them under ambient conditions for one week without losing viability and biological activities. This approach is particularly significant because it does not rely on the traditional cryopreservation method, which requires expensive equipment and complex procedures. It simplifies long-distance transportation of stem cells within temperature-mild areas and facilitates their therapeutic application without complex freezing and thawing processes. However, the spheropreservation technique involves preparing the hMSC to form spheroids using additional methods, such as the hanging-drop technique, which is tedious and complex to scale up. Moreover, the diameter of the cell spheroids needs to be about 250 μm, which is difficult to apply to uneven surfaces, for examples the of wounds of a patient having a diabetic foot (e.g., the spheroids may easily flow away or aggregate, and there may be uneven distribution of cells in a large and/or irregular area). Therefore, there is a need to develop a novel method of MSC transportation and delivery at ambient conditions that is scalable and easy to produce. Such a method should be adaptable in clinical settings, particularly in areas where access to cryopreservation facilities is limited.

### BRIEF SUMMARY

In one embodiment, a method of treating a wound on a patient is provided. The method comprises preparing a cell growth composition including MEM alpha, about 2-5% human platelet lysate or about 15-20% fetal bovine serum, about 0.5-1% L-glutamine, about 0.2-0.5% NEAA, about 0.1-1% antibiotics, and about 5-10% ng/mL bFGF. In one aspect, the method further comprises seeding cells on a scaffold immersed in the cell growth medium, growing cells to confluence on the scaffold by incubating the cells on the scaffold for at least 24 hours to form a cell sheet, preparing a cell transfer composition including about 10-50% MEM alpha (no Phenol Red), about 10-50% 1X DPBS or PBS, and about 15% fetal bovine serum or about 2-5% human platelet lysate or about 1-20% human serum albumin, transferring the sheet to the transfer medium contained in a transfer container, maintaining the viability of the cells on the cell sheet in the transfer medium at ambient conditions for at least 21 days. In another aspect, the live cells may be more than about 90% of all cells and dead cells may be less than about 5% of all cells when they are dissociated from the cells sheet for live/dead assay. In another aspect, the cell sheet may be directly applied to a wound on a patient.

In another embodiment, a method of generating a wound-healing device is provided. The method comprises preparing a cell growth composition including MEM alpha, about 15-20% fetal bovine serum or about 2-5% human platelet lysate, about 0.5-1% L-glutamine, about 0.2-0.5% NEAA, about 0.1-1% antibiotics, and about 5-10% ng/ml bFGF. In one aspect, the method further comprises seeding cells on a scaffold immersed in the cell growth medium, and growing cells to confluence on the scaffold by incubating the cells on the scaffold for at least 24 hours to form a cell sheet.

In another embodiment, a kit for wound healing or tissue engineering applications is provided. The kit comprises a transfer container comprising a cell sheet comprising cells seeded at about 2×10⁴ cells/cm² to about 7×10⁴ cells/cm² density on a scaffold, and a transfer medium comprising about 10-50% MEM alpha (no Phenol Red); about 10-50% 1X DPBS or PBS; and about 15% fetal bovine serum or about 2-5% human platelet lysate or about 1-20% human serum albumin. The cell sheet is immersed in the transfer medium configured to maintain cell viability and function at ambient conditions for at least 21 days.

### BRIEF DESCRIPTION OF THE DRAWINGS

FIGS. 1A-1F illustrate that adipose-derived stem cells (ADSC) may form a cell sheet with a nylon mesh and maintain viable cells for two weeks in ambient conditions. FIGS. 1A illustrates a cell sheet's side view, which appear white due to cell adhesion. FIGS. 1B illustrates a cell sheet's front view, which appear white due to cell adhesion. FIG. 1C illustrates how the cell sheet mimics an “artificial” skin sheet, a circular membrane about 2 cm in diameter, in its cultured state. FIG. 1D illustrates the cell sheet enclosed in a centrifuge tube. FIGS. 1E and 1F illustrate subsequent viability tests on the cell sheet.

FIGS. 2A-2F illustrate that human foreskin fibroblasts can form a cell sheet with a nylon mesh. FIG. 2A describes the morphology of the seed cells in the cell dish. FIG. 2B illustrates the morphology of the cell sheet when observed under a microscope (indicated by a dashed line circle). FIG. 2C shows a close-up view and surface texture of the cell sheet (within a dashed line frame, illustrating adjustable breathable micropores). FIG. 2D illustrates a complete form of an “artificial” skin sheet after being preserved in a unique solution for three weeks without degradation. Figure E illustrates cell survivability. Cells dissociated from the artificial skin (i.e., cell sheet) underwent three weeks of storage at ambient conditions, showing a high survival rate with more than 95±1% live cells (i.e., GFP-positive) and less than 3% dead cells (i.e., red-PI-positive) compared to the control which was stored for one week at ambient conditions with cell debris. The control had only 3% live cells (i.e., GFP-positive) and more than 92% dead cells (i.e., red-PI-positive). FIG. 2F illustrates that differences exist between the cell survival of artificial skin (i.e., cell sheet) and the control.

FIG. 3 illustrates directed differentiation of mesenchymal stem cell sheets into cartilage sheets. As shown in the control group, mesenchymal stem cells can adhere to the grid and grow as white sheets. After 18 days of directed differentiation in the experimental group, mesenchymal stem cells are differentiated into chondrocytes to form a cartilage sheet, which is stained blue by Alcian Blue.

FIG. 4 illustrates the application of artificial skin in a skin wound model using C57 mice. The artificial skin exhibits adherence to the wound site, accelerates wound closure, and does not induce inflammatory rejection.

FIG. 5 pictorially illustrates that compared to the control group, the animals treated with the artificial skin show enhanced wound healing, improved skin texture, and hair regrowth around the wound area.

FIG. 6 illustrates that compared to the control group, the animals treated with the artificial skin show an enhanced wound healing around the wound area when accounting for background intensity.

FIG. 7A illustrates how an artificial skin and a supporting base for a mask was prepared. FIG. 7B illustrates the step of placing the artificial skin at the center of a cotton pad. FIG. 7C illustrates the step of disinfecting the skin. FIG. 7D illustrates the step of directly applying the mask on the disinfected skin. FIG. 7E illustrates that upon removal the next morning, no redness or irritation was observed at the application site. FIG. 7F illustrates that the recovered artificial skin, when examined under a microscope, maintained a clear and intact structure without visible damage. FIG. 7G illustrates live-cell staining indicating that the viable cells remained attached to the fibrous substrate, exhibiting recovered artificial skin.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure. Accordingly, aspects and features of every embodiment may not be described with respect to each embodiment, but those aspects and features are applicable to the various embodiments unless statements or understandings are to the contrary.

As used herein, the term “about” or “approximately” refers to a quantity, level, value, number, frequency, percentage, dimension, size, amount, weight, or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicate the value plus or minus a range of 15%, 10%, 5%, or 1%. With respect to biological systems or processes, the term can mean within an order of magnitude, preferably within 5-fold, and more preferably within 2-fold, of a value. Unless otherwise stated, the term ‘about’ means within an acceptable error range for the particular value.

As used herein, the term “activated immune cells” refers to immune cells treated with one or more stimuli capable of inducing one or more alterations in the cell, for example, cellular alterations include, but are not limited to, metabolic, immunological, epigenetic, growth factor secreting, surface marker expression, and production and excretion of micro vesicles.

The term “administered” or “administering,” as used herein, refers to any appropriate method of providing a composition to an individual such that the composition has its intended effect on the patient. For example, one method of administering is by an indirect mechanism using a medical device such as, but not limited to, a catheter, applicator gun, syringe, gel matrix, or a 3D matrix containing one or more cell types, cell-derived products, and/or growth factors and/or antibiotics, etc. A second exemplary method of administering is by a direct mechanism such as, but not limited to, local tissue administration, oral ingestion, transdermal patch, topical, inhalation, suppository, etc.

As used herein, “allogeneic” refers to tissues or cells from another one or more different source or different individual of the same species, that, in a natural setting, are immunologically incompatible or capable of being immunologically incompatible.

As used herein, “autologous” refers to tissues or cells that are derived or transferred from the same individual (i.e., autologous blood donation; an autologous bone marrow transplant).

As used herein, “agent” refers to nucleic acids, cytokines, chemokines, transcription factors, epigenetics factors, growth factors, hormones, or a combination thereof, including whole cell lysate.

As used herein, “xenogeneic” refers to tissues or cells from a species different from the patient.

As used herein, “cell culture” is an artificial, in vitro system containing viable cells, whether quiescent, senescent, or (actively) dividing. In a cell culture, cells are grown and maintained at an appropriate temperature, typically a temperature of 37° C. and under an atmosphere typically containing oxygen and CO₂ at various concentrations. Culture conditions may vary widely for each cell type, though, and variation of conditions for a particular cell type can result in different phenotypes being expressed. The most commonly varied factor in culture systems is the growth medium or composition and oxygen concentration during culturing. Growth media can vary in concentration of nutrients, growth factors, and the presence of other components. The growth factors used to supplement media are often derived from animal blood, such as calf serum.

As used herein, “individual” refers to any human or animal. An individual may comprise any age of a human or non-human animal and therefore includes both adults and juveniles (i.e., children) and infants. It is not intended that the term “individual” connotes a need for medical treatment, therefore, an individual may voluntarily or involuntarily be part of experimentation, whether clinical or in support of basic science studies. The term “subject” or “individual” may be used interchangeably and refers to any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals.

As used herein, “transplantation” refers to the process of taking living tissue or cells and implanting it in another part of the body or into another individual.

As used herein, “treatment,” “treat,” or “treating” means a method of reducing the effects of a disease or condition. Treatment can also refer to a method of reducing the disease or condition itself rather than just the symptoms. The treatment may be any reduction from pre-treatment levels and may be, but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression, including a reduction in the severity of at least one symptom of the disease. For example, a disclosed method for reducing the immunogenicity of cells is considered to be a treatment if there is a detectable reduction in the immunogenicity of cells when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition but an improvement in the outlook of a disease or condition. In specific embodiments, treatment refers to the lessening in severity or extent of at least one symptom and may alternatively or in addition refer to a delay in the onset of at least one symptom.

The words “a” and “an” when used in the present disclosure in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein.

Throughout this specification, unless the context requires otherwise, the words “comprise,” “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory but that no other elements are optional and may or may not be present depending upon whether or not they affect the activity or action of the listed elements.

Reference throughout this specification to “one embodiment,” or “one aspect”, “an embodiment,” or “an aspect”, or combinations thereof means that a particular aspect, feature, structure, or characteristic described in connection with an embodiment is included in at least one embodiment of the present disclosure. Thus, the appearances of the foregoing phrases in various places throughout this specification are not necessarily all referring to the same embodiment or same aspect. Furthermore, the particular features, structures, or characteristics may be combined in any suitable manner in one or more embodiments or aspect.

The terms “reduce,” “inhibit,” “diminish,” “suppress,” “decrease,” “prevent,” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The present disclosure is directed to compositions, methods, and systems for the generation, culture, maintenance, preservation, and transportation of cell sheets.

The cell sheets may include one or more types of cells. In some embodiments, the cell sheet may include more than one type of cell. For example, the cell sheet may include a first type of cell and a second type of cell.

In one embodiment, the cells may be mesenchymal stem cells. In some embodiments, the cells may be autologous mesenchymal stem cells. In some embodiments, the cells may be allogeneic mesenchymal stem cells. In some embodiments, the cells may be xenogeneic mesenchymal stem cells. In some embodiments, the cells may be adipose-derived mesenchymal stem cells, bone marrow mesenchymal stem cells, placental mesenchymal stem cells, omental tissue-derived mesenchymal stem cells, umbilical cord blood-derived mesenchymal stem cells, pluripotent stem cells-derived mesenchymal stem cells, fetal stem cells, neonatal stem cells, or adult stem cells. In some embodiment, the cell sheet further includes mesenchymal stem cells derived material.

In one embodiment, the cells may be keratinocytes, myofibroblasts, chondrocytes, or osteocytes. In some embodiment, the cells may be epithelial cells, keratinocytes, basal epithelial cells, myofibroblasts, cardiac cells, organ-isolated primary cells, or mixtures thereof. In one embodiment, the cells may be derived from various tissues or organs, including but not limited to skin, heart, blood vessels, bone marrow, skeletal muscle, liver, pancreas, brain, and foreskin, which may be obtained by biopsy or upon autopsy.

In one embodiment, the cell sheet may include a confluent cell layer on a scaffold. In some embodiments, the scaffold may comprise an inert material. In some embodiments, the scaffold may be silk or silk derivatives, hydrogel or hydrogel derivatives, or a nylon mesh. In one embodiment, the cells may spontaneously attach to the scaffold. In some embodiments, ultra-low attachment culture plates or glass culture flasks may be used with cells at a high density.

In one embodiment, the scaffold may have a pore size of about 25 μm to about 500 um, including any size or range comprised therein. For example, the scaffold may have a pore size of about 25 μm to about 40 μm, about 30 μm to about 50 μm, about 40 μm to about 70 μm, about 50 μm to about 80 μm, about 70 μm to about 100 μm, about 100 μm to about 200 μm, about 200 μm to about 300 μm, about 300 μm to about 400 μm, or about 400 μm to about 500 μm.

In one embodiment, the scaffold may have a thickness of about 10 μm to about 500 μm, including any size or range comprised therein. For example, the scaffold may have a thickness of about 60 μm to about 70 μm, about 10 μm to about 50 μm, about 50 μm to about 100 μm, about 100 μm to about 200 μm, about 200 μm to about 300 μm, about 300 μm to about 400 μm, or about 400 μm to about 500 μm.

In one embodiment, the scaffold may have an area of about 0.1 cm² to about 1 m², including any size or range comprised therein. For example, the scaffold may have an area of about 0.1 cm² to about 1 cm², about 1 cm² to about 10 cm², about 10 cm² to about 100 cm², about 100 cm² to about 1000 cm², about 1000 cm² to about 1 m².

In one embodiment, a method of generating a cell sheet may comprise preparing a cell growth composition, seeding cells at a threshold density on a scaffold immersed in the cell growth composition, and growing cells to confluence on the scaffold by incubating the cells on the scaffold for at least 24 hours to form a cell sheet. In some embodiments, the cells were grown to confluence on the scaffold by incubating the cells on the scaffold for about 24 hours to about 72 hours, including any time or range comprised therein. In some embodiments, the cell sheet may not include the scaffold.

In one embodiment, the cells may attach to the scaffold to form the cell sheet in a 37° C. incubator with about 5% carbon dioxide and greater than about 80% humidity. In one embodiment, the cells may attach to the scaffold to form the cell sheet under different conditions. Any suitable conditions that achieve confluence and facilitate cell attachment to the scaffold is contemplated herein.

The threshold density may range from about 2×10⁴ cells/cm² to about 7×10⁴ cells/cm² including any density or range comprised therein. For example, the threshold density may be between about 2×10⁴ cells/cm² to about 3×10⁴ cells/cm², between about 3×10⁴ cells/cm² to about 4×10⁴ cells/cm², between about 4×10⁴ cells/cm² to about 5×10⁴ cells/cm², between about 5×10⁴, cells/cm² to about 6×10⁴ cells/cm², or between about 6×10⁴ cells/cm² to about 7×10⁴ cells/cm².

The cell growth composition may include MEM alpha, about 15 to 20% fetal bovine serum or about 2-5% human platelet lysate, about 0.2 to 1% L-glutamine, about 0.2 to 0.5% NEAA, about 0 to 1% antibiotics, and about 5 to 15 ng/mL bFGF. In some embodiments, the cell growth composition may include one or more of epidermal growth factor, leukemia inhibitory factor, insulin-like growth factor, angiopoietin, and vascular endothelial growth factor. In some embodiments, the cell growth composition may be xenogeneic compound free. In some embodiments, the cell growth composition may be free of non-human animal-derived materials including but not limited to fetal bovine serum. In some embodiments, the cell growth composition may include a component that may facilitate the reduction of immunogenicity of the cell sheet. In some embodiments, the cell growth composition may include human platelet-rich plasma, platelet lysate, umbilical cord blood serum, autologous serum, and one or more defined cytokines, such as one or a combination of fibroblast growth factor, epidermal growth factor, leukemia inhibitory factor, insulin-like growth factor, angiopoietin, and vascular endothelial growth factor, or other similar components.

In one embodiment, the method of generating a cell sheet may be an in vitro method. In one embodiment, the method of generating a cell sheet may include growing cells to a confluent cell layer configured to inhibit and/or reduce cell proliferation and/or cell metabolism rates. In one embodiment, the method of generating a cell sheet may include growing cells to a confluent cell layer configured to reduce oxygen consumption and nutrient consumption resulting from cell-cell contact inhibition.

In one embodiment, the cell sheet may be transferred to a site of application immersed in a cell transfer composition comprised in a transfer container. The cell transfer composition may have no biological toxicity. The cell transfer composition may be produced for high density and large-scale storage.

In some embodiments, the cell transfer composition may include about 10% to about 50% MEM alpha (no Phenol Red), about 10% to about 50% 1X DPBS or PBS, and about 15% fetal bovine serum, about 2% to about 5% human platelet lysate, 1% non-essential amino acids, 5% L-glutamine, and/or 1% to about 20% human serum albumin.

The cell sheet immersed in the cell transfer composition may not need subsequent separation and purification, and the entire storage and transportation process may not require specific temperature and humidity. Therefore, the cell sheet may be directly used at the application site.

In some embodiments, the cell transfer composition may ensure basic energy, nutrition maintenance needs, and suitable pH for cell survival and reduce any shear stress damage caused by bumps during transportation. In some embodiments, the cell transfer composition may not require any specific temperature and gas maintenance equipment.

In some embodiments, the cell transfer composition may be removed at a final destination by centrifugation to collect a cell sheet. The collected cell sheet may be used directly for assays and/or treatment.

In one embodiment, the cell transfer composition further comprises a tackifier that reduces shear on cells during transportation. The tackifier may be a reagent or component that is added to the composition to increase the viscosity and/or stickiness of the composition. In some embodiments, the tackifier may be a food-grade additive that can increase the viscosity of the cell transfer composition. In some embodiments, the tackifier may be methylcellulose, gelatin, or hydrogel. In some embodiments, the cell transfer composition may include between about 0.2% to about 5% of the tackifier, including any percentage or range comprised therein. For example, the cell transfer composition may include between about 0.2% to about 0.5%, between about 0.5% to about 1%, between about 1% to about 2%, between about 2% to about 3%, between about 3% to about 4%, or between about 4% to about 5% of the tackifier.

In one embodiment, the transfer container may comprise a chemically inert material. In some embodiments, the transfer container may comprise a plastic. In some embodiments, the transfer container may be transparent. In some embodiments, the transfer container may be sterilized. In some embodiments, the transfer container may comprise polyethylene, polypropylene, or melamine. In one embodiment, the cell sheet may be transferred under ambient conditions. In one embodiment, the cell sheet may be transferred under cryopreservation.

In one embodiment, the cell sheet has a viability of more than 90% after being immersed in the cell transfer composition under ambient conditions for at least two weeks. In one embodiment, the cell sheet has a viability of more than 90% after being immersed in the cell transfer composition under ambient conditions for at least three weeks. In some embodiments, the cell sheet has a viability of more than 90% after being immersed in the cell transfer composition under ambient conditions for about 14 to 25 days, including any number of days or range comprised therein. In some embodiments, the percentage of dead cells on the cell sheet is less than about 5% after being immersed in the cell transfer composition for at least two weeks. In one embodiment, the cell sheet comprises cells with biological activity after being immersed in the cell transfer composition for at least two weeks.

In some embodiments, stem cells or stem cell-derived materials may be modified before they are used in the targeted tissue or organ. Such modification may be a chemical, viral, physical, or epigenetically activated modification. Such modification may occur after exposure to conditions not typically found in the body. In some embodiments, immune cells such as neutrophils, macrophages, tissue-specific mesenchymal stem cells, keratinocytes, basal epithelial cells, myofibroblasts, or their derivatives may be modified or activated before their exposure to the site of usage. In one embodiment, the cell sheet may recruit immune cells and/or stem cells at the site of usage.

In one embodiment, the cell sheet may be activated by a chemical agent, a biological agent, a cytokine, a chemokine, a growth factor, RNA, micro-RNA, RNAi, DNA, a virus, an exosome, or a pharmaceutical composition before being used for the treatment of a patient. In some embodiments, the cell sheet may be modified by a chemical agent, a biological agent, a cytokine, a chemokine, a growth factor, RNA, micro-RNA, RNAi, DNA, a virus, an exosome, or a pharmaceutical composition during the preparation, culturing, maintenance, and or preservation of the mesenchymal stem cell sheet. In one embodiment, one or more cell types comprised in the cell sheet may be modified by a chemical agent, a biological agent, a cytokine, a chemokine, a growth factor, RNA, micro-RNA, RNAi, DNA, a virus, an exosome, or a pharmaceutical composition.

In one embodiment, the cell sheet may be used in disease modeling, clinical training, clinical research, and/or therapeutic applications for humans and animals. In some embodiments, the cell sheet may be used for tissue regeneration, immune modulation, tissue repair, wound healing, or as a targeted tool for cell migration. In some embodiments, the cell sheet may be used to treat a wound or a burn in a patient. In some embodiments, the cell sheet may be used for cell therapy for immune diseases and degenerative diseases, cell transplantation for tissue and organ damage, or drug screening.

In one embodiment, more than one cell sheet may be used simultaneously or consecutively for tissue regeneration, immune modulation, tissue repair, wound healing, or as a targeted tool for cell migration. In some embodiments, more than one cell sheet may be used to treat a wound or a burn in a patient. In some embodiments, the cells seeded on more than one sheet used simultaneously or consecutively may be the same. In some embodiments, the cells seeded on more than one sheet used simultaneously or consecutively may be different.

In one embodiment, the cell sheet may be used as a device for extended-time release of mesenchymal stem cells and/or mesenchymal stem cell-derived materials in vivo in a patient. It may also be used as a device for extended-time release for tissue regeneration, immune modulation, tissue repair, and wound healing or as a targeted tool for cell migration. In one embodiment, the cell sheet may be used as a human skin contact device such as a face mask or bandage.

In one embodiment, the cells in the cell sheet may differentiate into any type of somatic functional cell, including, but not limited to, muscle cells, fat cells, skin cells, bone cells, and cartilage cells. Mesenchymal stem cell-derived somatic functional cell sheets can be used for disease treatment, such as wound healing, bone fractures, and osteoarthritis.

In one embodiment, the cell sheet may be used as an overnight facial mask, allowing the cells to sense the user's skin microenvironment and directly exert anti-inflammatory, desensitizing, and anti-aging effects.

In one embodiment, a method of using the cell sheets may comprise co-administration of universal donor mesenchymal stem cell sheets, gene-modified cell sheets, xenogeneic cell sheets, and/or cell sheets with autologously modified with one or more agents that stimulate and maintain a desired immune response, tissue repair, and or tissue regeneration. In one embodiment, a method of using the cell sheets may comprise co-administration of universal donor mesenchymal stem cell sheet and mesenchymal stem cell sheet-derived materials with one or a combination of chemokines and cytokines and growth factors. In one embodiment, the universal donor mesenchymal stem cell sheet may have been treated under conditions to reduce immunogenicity for tissue repair and/or tissue regeneration.

In one embodiment, a kit for wound healing or tissue engineering applications is provided. The kit comprises a transfer container comprising a cell sheet comprising cells seeded at between about 2×10⁴ cells/cm² to 7×10⁴ cells/cm² density on a scaffold, and a transfer medium comprising about 10% to about 50% MEM alpha (no Phenol Red); 10-50% 1X DPBS or PBS; and 15% fetal bovine serum or 2% to about 5% human platelet lysate or about 1% to about 20% human serum albumin. The cell sheet may be immersed in the transfer medium configured to maintain cell viability at ambient conditions for at least 15 days.

In some embodiments, the cell viability of the cells in the kit may be about from about 1 to 21 days. In some embodiments, the cells in the kit may be mesenchymal stem cells. In some embodiments, the cells in the kit may be autologous. In some embodiments, the cells on the cell sheet in the kit may be allogeneic. In some embodiments, the cells in the kit may be xenogeneic. In some embodiments, the cells on the cell sheet in the kit may be adipose-derived mesenchymal stem cells, bone marrow mesenchymal stem cells, placental mesenchymal stem cells, omental tissue-derived mesenchymal stem cells, or umbilical cord blood-derived mesenchymal stem cells. In some embodiments, the cells on the cell sheet in the kit may be activated or inactivated keratinocytes, myofibroblasts, chondrocytes, or osteocytes. In some embodiments, the cells on the cell sheet in the kit may be epithelial cells, immune cells, neutrophils, macrophages, keratinocytes, basal epithelial cells, myofibroblasts, organ-derived primary cells, or mixtures thereof. In some embodiments, the cell sheet in the kit may further comprise mesenchymal stem cell-derived material.

In some embodiments, the cells on the cell sheet in the kit may be configured to form a monolayer such that cell contact inhibition results in reduced cell proliferation and metabolism rates. In some embodiments, the cell sheet in the kit may be configured to comprise cells with reduced oxygen consumption and nutrient consumption.

In some embodiments, the transfer medium in the kit may further comprise a tackifier to reduce shear stress on the cells during transportation. In some embodiments, the tackifier may be a food-grade additive that can increase the viscosity of the transfer medium. In some embodiments, the tackifier may be methylcellulose, gelatin, or hydrogel. In some embodiments, the transfer medium may comprise about 0.2% to about 5% of the tackifier.

In some embodiments, the scaffold of the cell sheet in the kit may have an area of about 0.1 cm² to about 1 m². In some embodiments, the scaffold of the cell sheet in the kit may have a thickness of about 60 μm to about 120 μm. In some embodiments, growing cells to confluence on the scaffold in the kit may comprise incubating the cells on the scaffold for about 24 to 72 hours.

In some embodiments, the scaffold of the cell sheet in the kit may be an inert material. In some embodiments, the scaffold of the cell sheet in the kit may comprise nylon, silk, or hydrogel. In some embodiments, the scaffold of the cell sheet in the kit may comprise nylon, silk, or hydrogel. In some embodiments, the scaffold of the cell sheet in the kit may have a pore size of about 50 μm to about 500 μm.

In some embodiments, the cells on the cell sheet in the kit may be used by activating the cell sheet through a chemical agent, RNA, micro-RNA, or RNAi, DNA, a virus, or an exosome.

In some embodiments, the transfer container in the kit may comprise a chemically inert material, such as polyethylene, polypropylene, or melamine. The kit may further comprise one or more of an antiseptics, bandages, gauze, gloves, scissors, and ointments.

In some embodiments, the cell sheet may be used as a facial mask. Beyond its use as a facial mask, this technology also holds potential for broader applications, such as intimate care products incorporating live cells-including patches, tampons, and hemorrhoid treatments.

### EXAMPLES

### Example 1—Human Adipose-Derived Mesenchymal Stem Cells

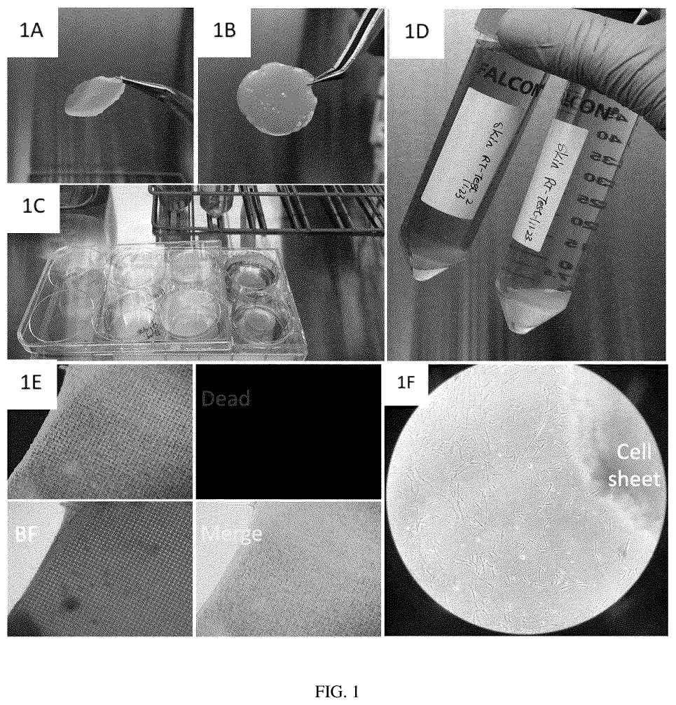

Human adipose-derived mesenchymal stem cells (ADSC) sheets are highly viable after being stored at room temperature for two weeks. FIG. 1 illustrates that adipose-derived stem cells (ADSC) may form a cell sheet with a nylon mesh and maintain viable cells for two weeks in ambient conditions. ADSCs were grown in a normal cell culture environment (37° C., 5% carbon dioxide, 90% humidity) to 80% confluency for experiments. The medium and cells were removed and washed twice with 1X PBS. The cells were dissociated with 0.25% trypsin for 3 minutes. Stem cell medium (low glucose DMEM, 10% fetal bovine serum, 1% non-essential amino acids, 5% L-glutamine) was used to collect the cells. The cell concentration was adjusted to 3×10⁶ cells/mL and incubated with a nylon mesh to prepare the stem cell sheet in an ultralow attachment plate. The culture plate was placed in a standard cell culture environment for three days. The stem cell sheet was collected from the plate and centrifuged at 600 rpm for 3 minutes to remove the supernatant. FIGS. 1A and 1B illustrate a cell sheet's side and front views, which appear white due to cell adhesion. The thickness of the sheet is about 80 micrometers. FIG. 1C illustrates the cell sheet mimics an “artificial” skin sheet, a circular membrane about 2 cm in diameter, in its cultured state. A preservation matrix was added to suspend the cell sheet million in a 50 mL tube. The cells were added to a 40 mL plastic centrifuge tube, sealed with parafilm, and stored in the dark at room temperature (about 22° C.) for two weeks. FIG. 1D illustrates the cell sheet enclosed in a centrifuge tube. These tubes were stored at ambient condition (i.e., room temperature) for two weeks.

The supernatant was removed on the 15th day and the cell sheet was transferred to a cell culture plate. 1.5 mL medium was into the well with cell sheet and cultures overnight for attachment. The cells were tested, applied, or returned to the standard culture environment to continue culturing. The cell sheet was attached to the cell culture plate, and the ADSCs migrated out of the cell sheet overnight. The cell sheet showed a 100% survival rate with AO/PI staining and showed normal cell morphology, cell proliferation, and migration ability. FIGS. 1E and 1F illustrate subsequent viability tests on the cell sheet. It shows nearly 100% survival in situ with AO/PI staining, indicated by a predominance of green cells (AO-positive, representing high metabolic activity, upper left) and an almost complete absence of red (PI-positive, dead, upper right) cells. After being stored for two weeks under ambient condition (i.e., room temperature), the cell sheet demonstrated rapid adherence and cell growth on the surface of the cell culture dish. The stem cells within the cell sheet could migrate from the cell sheet to the surface of the culture dish within two days. Such migration may be indicative of stem cell release from the sheet and migration of the stem cells into the wound.

### Example 2—Human Fibroblast Cells

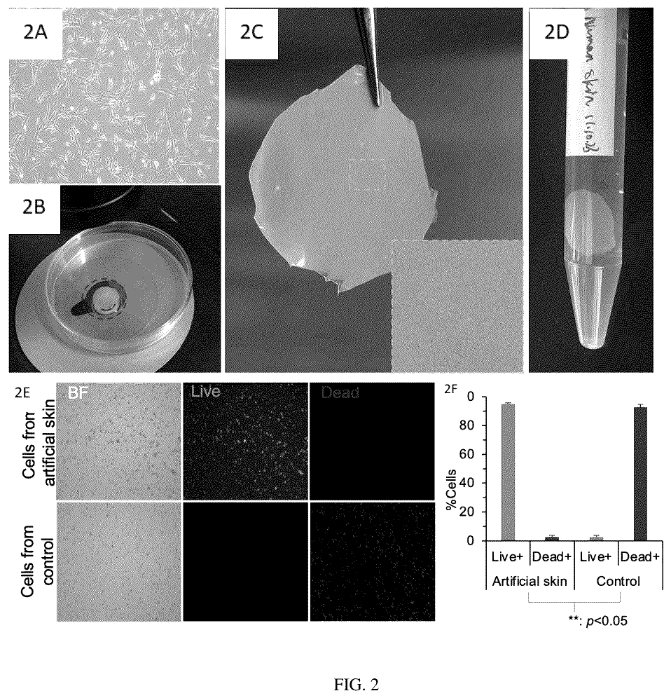

Human fibroblast sheet-formed artificial skin was kept at room temperature for three weeks and was highly viable after reattachment in culture (as shown in FIG. 2). One strain of human fibroblast cells was derived from the human foreskin. The skin fibroblasts were used for validation experiments. Cells were grown in a normal cell culture environment (37° C., 5% carbon dioxide, 90% humidity) to 80% confluency for experiments. The medium and cells were removed and washed twice with 1X PBS. The cells were dissociated with 0.25% trypsin for 3 minutes. Stem cell medium (low glucose DMEM, 10% fetal bovine serum, 1% non-essential amino acids, 5% L-glutamine) was used to collect the cells. The cell concentration was adjusted to 3×10⁶ cells/mL and incubated with a nylon mesh to prepare the stem cell sheet in an ultralow attachment plate. The culture plate was placed in a standard cell culture environment for three days. The stem cell sheet was collected from the plate and centrifuged at 600 rpm for 3 minutes to remove the supernatant. A preservation matrix was added to suspend the cell sheet million in a 50 mL tube. The cells were added to a 40 mL plastic centrifuge tube, sealed with parafilm, and stored in the dark at room temperature (about 22° C.) for three weeks. The 3×10⁶ cell in 15 mL medium suspension was kept in the tube as a control. The supernatant was removed on the 21^{st} day and the cell sheet was transferred to a cell culture plate. 1.5 mL medium was into the well with cell sheet and cultures at normothermia (i.e., 37° C., 5% CO₂ and 90% humidity environment) overnight for attachment. The cell sheet showed a 100% survival rate with AO/PI staining and showed normal cell morphology, cell proliferation, and migration ability.

### Example 3: Cartilage Differentiation

Human mesenchymal stem cell sheets may directly differentiate to be the cartilage sheet. ADSCs were grown in a typical cell culture environment (37° C., 5% carbon dioxide, 90% humidity) to 80% confluency for experiments.

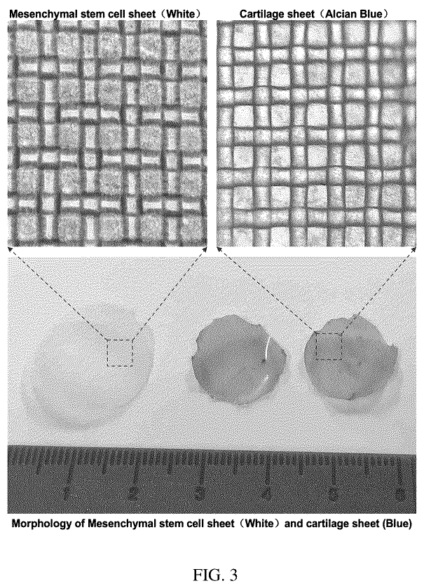

Cells were grown in a normal cell culture environment (37° C., 5% carbon dioxide, 90% humidity) to 80% confluency for experiments. The medium and cells were removed and washed twice with 1X PBS. The cells were dissociated with 0.25% trypsin for 3 minutes. Stem cell medium (low glucose DMEM, 10% fetal bovine serum, 1% non-essential amino acids, 5% L-glutamine) was used to collect the cells. The cell concentration was adjusted to 3×10⁶ cells/mL and incubated with a nylon mesh to prepare the stem cell sheet in an ultralow attachment plate. The culture plate was placed in a typical cell culture environment for three days. The ADSC sheet was rinsed with 1X DPBS twice. The ADSC sheet was placed in one well of the ultralow attachment dish (6-well plate) and a chondrocyte differentiation medium (Gibco, #A1007101) was added. The medium was changed every other day. On the 20th day, the supernatant was removed, and the cell sheet was rinsed with 1X DPBS twice. The 1X DPBS was removed, and the cell sheet was fixed with 4% PFA and stained with 1% Alcian Blue solution. The human ADSC sheets were differentiated into cartilage sheets within 20 days. With its glycosaminoglycans, the differentiated chondrocyte can be identified by Alcian blue staining. The morphology of the ADSC differentiation cartilage sheet and the results of chondrocyte identification with Alcian blue are indicated in FIG. 3.

### Example 4: Cell Sheet for Wound Healing Treatment

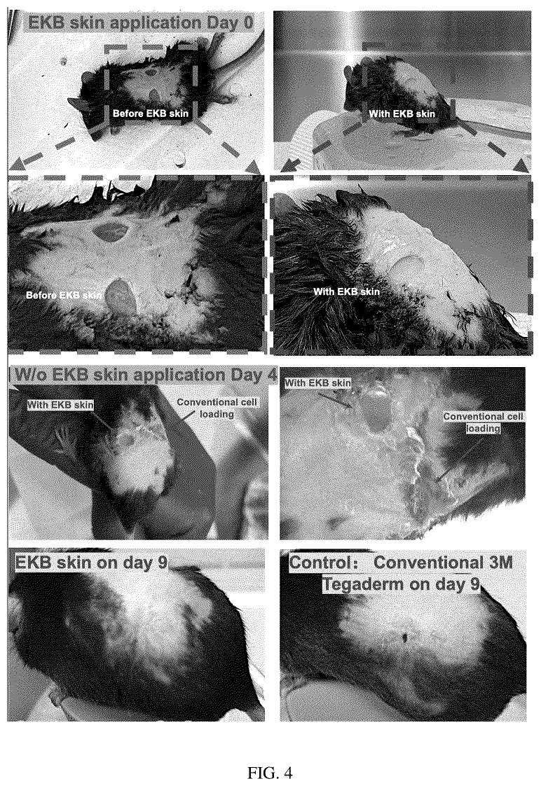

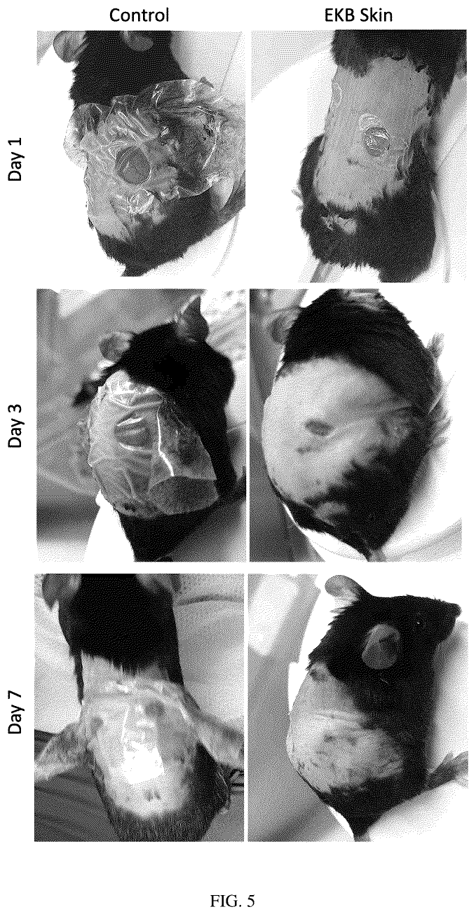

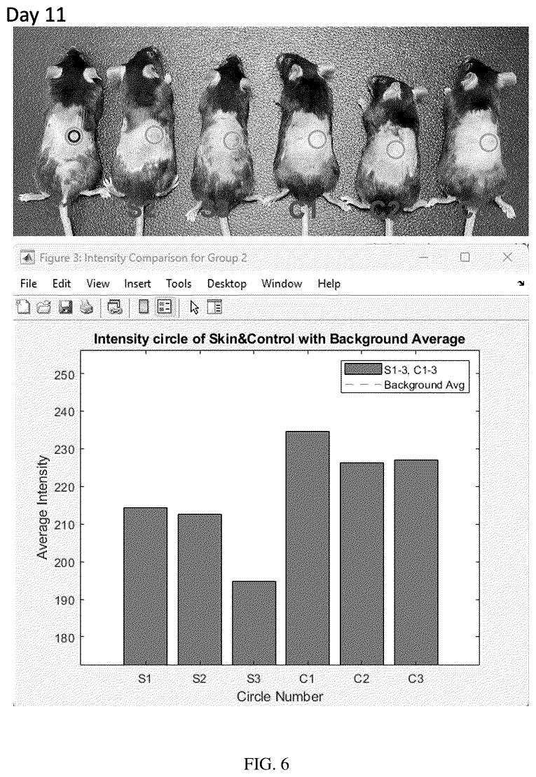

Cell sheets were used for wound healing treatments in C57 mouse. Briefly, the hair on the back of mice were removed with cream and the moped with 70% ethanol to sterilize the skin. A puncher was used to make two wounds about 6 mm diameter. Cell sheets with the same wound size were used to cover the surface of the wound and then protected by Tega derm or liquid bandage. The wound was allowed to heal through 12 days. FIG. 4 illustrates the application of artificial skin (EKB Skin) in a skin wound model using C57 mice. As shown, the artificial skin exhibits excellent adherence to the wound site, accelerates wound closure, and does not induce inflammatory rejection. Compared to the control group, the animals treated with the artificial skin show enhanced wound healing, improved skin texture, and hair regrowth around the wound area, as shown in FIGS. 5 and 6.

The results above illustrate the advantages of directly applying a cell sheet on the top of the wound (i.e., on a patient with diabetic foot). The method comprises tailoring a scaffold to fit the area of the wound. The method further comprises allowing stem cells to be attached to the tailored scaffold and are cultured under the ordinary cell culture condition to form the stem cells sheet. The method further comprises storing the cell sheet in the cell preservation medium after about 24-28 hours and delivering the cell sheet to the location where it is needed. Such cell sheets may be evaluated in mouse wound healing models. After applying the cell sheet, a sterile dressing may be used to cover the wound.

### Example 5: Cell Sheet for Human Skin Contact and Application as a Facial Mask

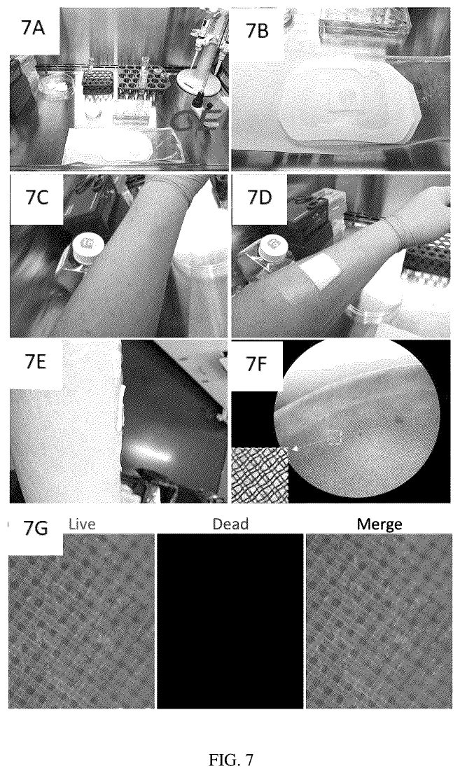

An artificial skin and a supporting base for the mask was prepared (Tegaderm with a cotton pad, FIG. 7A). The artificial skin was placed at the center of the cotton pad (FIG. 7B). During application, the skin was disinfected (FIG. 7C), and the mask was applied directly (FIG. 7D). The entire process of using the artificial skin may be completed by a single user.

No discomfort was reported throughout the night while the user was wearing the artificial skin mask. Upon removal the next morning, no redness or irritation was observed at the application site (FIG. 7E). The recovered artificial skin, when examined under a microscope, maintained a clear and intact structure without visible damage (FIG. 7F). Live-cell staining showed that the viable cells remained attached to the fibrous substrate, exhibiting strong activity (green fluorescence), with no signs of dead cells (no red fluorescence observed) (FIG. 7G). These results indicate that the artificial skin mask may remain viable after about 14 hours of contact with human skin.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The examples are provided by way of illustration only and not by way of limitation. Those skilled in the art will readily recognize a variety of noncritical parameters that could be changed or modified to yield essentially similar results. References provided throughout are meant to be incorporated by reference only with regard to such background teaching that they refer to.

What is claimed:

1. A method of treating a wound on a patient in need thereof, comprising:
(a) preparing a cell growth composition comprising:
(i) MEM alpha;
(ii) about 15% to about 20% fetal bovine serum or about 2% to about 5% human platelet lysate;
(iii) about 0.5% to about-1% L-glutamine;
(iv) about 0.2% to about 0.5% NEAA;
(v) about 0.1% to about-1% antibiotics; and
(vi) about 5% to about-10% ng/mL bFGF;
(b) seeding one or more cells on a scaffold immersed in the cell growth composition;
(c) forming a cell sheet;
(d) transferring the sheet to a transfer medium;
(e) maintaining viability and function of the cells on the cell sheet at ambient conditions for at least 21 days; and
(f) applying the sheet to a wound on the patient.

2. The method of claim 1, wherein the transfer medium comprises:
(a) about 10% to about 50% MEM alpha;
(b) about 10% to about 50% 1X DPBS or PBS; and
(c) about 15% fetal bovine serum, about 2% to about 5% human platelet lysate, or about 1% to about 20% human serum albumin.

3. The method of claim 1, wherein the one or more cells are mesenchymal stem cells, epithelial cells, neutrophils, macrophages, keratinocytes, basal epithelial cells, myofibroblasts, or isolated primary cells.

4. The method of claim 3, wherein the mesenchymal stem cells are autologous, allogenic, or xenogeneic.

5. The method of claim 1, wherein the cells are seeded at a density of about 2×10⁴ cells/cm² to 7×10⁴ cells/cm² on the scaffold.

6. The method of claim 1, wherein the scaffold is made of an inert material.

7. The method of claim 1, wherein the scaffold comprises an area of about 0.1 cm² to about 1 m².

8. The method of claim 1, wherein the scaffold comprises a thickness of about 60 μm to about 120 μm.

9. The method of claim 1, wherein the scaffold comprises a pore size of about 10 μm to about 500 μm.

10. The method of claim 1, wherein the cell sheet is configured to have a monolayer of cells.

11. The method of claim 1, wherein the cell growth composition further comprises one or more of: epidermal growth factor, leukemia inhibitory factor, insulin-like growth factor, angiopoietin, or vascular endothelial growth factor.

12. The method of claim 1, wherein the transfer medium further comprises a tackifier comprising one or more of methylcellulose, gelatin, or a hydrogel.

13. The method of claim 12, wherein the tackifier is present in an amount of between about 0.2% to about 5%.

14. The method of claim 1, wherein the sheet is applied in combination with one or more of an antibiotic, a pain medication, or a growth factor.

15. The method of claim 1, wherein the sheet is a first sheet.

16. The method of claim 15, wherein the first sheet is applied in combination with a second sheet.

17. The method of claim 15, wherein the cells seeded on the first sheet are different from cells seeded on the second sheet.

18. A method of generating a wound healing device comprising:
(a) preparing a cell growth composition comprising:
(i) MEM alpha;
(ii) about 15% to about 20% fetal bovine serum or about 2% to about 5% human platelet lysate;
(iii) about 0.5% to about-1% L-glutamine;
(iv) about 0.2% to about 0.5% NEAA;
(v) about 0.1% to about-1% antibiotics; and
(vi) about 5% to about-10% ng/mL bFGF;
(b) seeding cells on a scaffold immersed in the cell growth medium.

19. The method of claim 18, wherein the wound healing device is used as a human skin contact device.

20. A kit comprising the wound healing device of claim 18, further comprising one or more of a transfer medium, an antiseptic, bandages, gauze, gloves, scissors, or ointments.
